(19) United States
(12) Patent Application Publication
(10) Pub. No.: US 2011/0247612 A1
(43) Pub. Date: Oct. 13, 2011

(54) OXYGEN AND AROMATHERAPY DELIVERY APPARATUS FOR A MASSAGE TABLE

(76) Inventor: Micheal Joseph DeSalvo, Highland Beach, FL (US)

(21) Appl. No.: 13/085,772

(22) Filed: Apr. 13, 2011

Related U.S. Application Data
(60) Provisional application No. 61/342,408, filed on Apr. 13, 2010.

(51) Int. Cl. A61M 11/00

(52) U.S. Cl. 128/200.23

(57) ABSTRACT

An apparatus includes a device that has an interior chamber, an opening at a bottom end for connection to a flexible hose, and an enlarged upper end with an annular cloth cover. A central opening in the cover is surrounded by an elastomeric band that stretches to enlarge the central opening, thereby allowing the cover to fit over the face cradle of a conventional massage table so that the chamber of the device is positioned below the face cradle. An opposite end of the flexible hose connects to an oxygen or aroma generating source. Oxygen and/or the aromas are delivered through the bottom end of the delivery device and into the chamber for breathing by a person lying face down on the massage table with their face supported in the face cradle, thereby allowing the person to receive an oxygen therapy and/or aromatherapy treatment during a massage.

This nonprovisional application is based on provisional patent application Ser. No. 61/342,408 filed on Apr. 13, 2011.

### BACKGROUND OF THE INVENTION

1. Field of the Invention

The present invention is directed to an aromatherapy and oxygen delivery apparatus, and, more particularly to an apparatus that attaches to the face cradle of a conventional massage table for delivering oxygen and/or pleasant aromas to a person to breathe while lying face down on a massage table during a massage.

2. Discussion of the Related Art

It is well known that massage therapy is highly beneficial for health and well being. Typically, massage therapy is administered on a massage table that is specifically designed to support a person while lying down in a comfortable horizontal position and at an ideal height for the massage therapist. During the massage therapy session, the person receiving the massage is required to lie face down for at least a portion of the session so that the therapist can massage the shoulder, back and leg muscles. While lying in the face down position, a face cradle is often attached to the end of the massage table in order to comfortably support the person's head. The face cradle is generally U-shaped and supports the forehead and sides of the face so that the person's nose and mouth are unobstructed, thereby allowing the person to breathe through the open area of the cradle. Other massage tables have a built-in opening for receiving the user's face when lying face down.

In recent years, the benefits of oxygen therapy and aromatherapy have been well publicized. In response to an increased demand for these highly beneficial treatments, many spas are now offering oxygen therapy and aromatherapy to customers. These treatments are normally administered while the person is seated in a comfortable lounge chair in a calm and relaxing environment. Customers visiting a spa for their massage therapy or other spa treatments may be offered the opportunity to receive oxygen therapy or aromatherapy in a designated room or area having one or more lounge chairs. To receive oxygen therapy, customers are required to wear a mask or a hose that wraps over the ears and connects to a fitting that inserts in the nostrils. These devices are generally uncomfortable and annoying to many people. The oxygen therapy or aromatherapy session is usually offered as an additional spa service. Customers may pay an additional expense for this service that requires the customer to spend more time in the spa. Some customers may not have the additional time to spend at the spa in order to receive multiple spa services that may require several hours. Accordingly, there remains a need for a device or apparatus that is adapted to deliver oxygen therapy and/or aromatherapy to a person at the same time the person is receiving a massage therapy treatment. This will allow the person to enjoy the benefits of oxygen therapy and/or aromatherapy during a massage, without requiring additional time to receive separate treatments and without having to wear a mask or other device that contacts the face. This also allows a spa or massage therapy business to offer additional services, and particularly oxygen therapy or aromatherapy, to customers while they enjoy a massage. Moreover, it is believed that an oxygen treatment, alone or in combination with aromatherapy, may help a person relax during a massage while delivering more oxygen to the muscle tissue to enhance the massage treatment.

### OBJECTS AND ADVANTAGES OF THE INVENTION

Considering the foregoing, it is a primary object of the present invention to provide an apparatus for use with a massage table in order to deliver oxygen and/or an aromatherapy treatment to a person lying face down on the massage table while receiving a massage treatment without the need of wearing a mask or other device that touches the face.

It is still a further object of the present invention to provide an aromatherapy and oxygen delivery apparatus that includes a device for easy attachment to the massage table, and wherein the device connects to a supply of oxygen and/or an aroma generating source for aromatherapy in order to deliver oxygen and/or the beneficial aromas to a person lying face down on the massage table, thereby allowing the person to easily breathe the oxygen and/or aromas while receiving a massage treatment.

It is still a further object of the present invention to provide an aromatherapy and oxygen delivery apparatus for use with a massage table that will allow a spa or massage therapy business to offer oxygen delivery and/or aromatherapy to customers during a massage.

These and other objects and advantages of the present invention are more readily apparent with reference to the detailed description and accompanying drawings.

### SUMMARY OF THE INVENTION

One preferred embodiment of the present invention is directed to an apparatus that includes a device that has an interior chamber, an opening at a bottom end for connection to a flexible hose, and an enlarged upper end with a washable or disposable cloth cover. A central opening in the cover is surrounded by an elastomeric band that stretches to enlarge the central opening, thereby allowing the cloth cover to fit over the face cradle of a conventional massage table so that the chamber of the device is positioned below the face cradle. The flexible hose connects to an oxygen or aroma generating source. In the one preferred embodiment, the device includes a lower portion that is in the shape of a funnel which may be made of plastic or another rigid or semi-rigid material or a fabric. The device may be made to be easily cleaned and disinfected after each use. Alternatively, the device may be disposable so that a new sterile device is replaced on the face cradle before each use. Oxygen and/or the pleasant aromas of essential oil are delivered through the bottom end of the funnel shaped lower portion for breathing by a person lying face down on the massage table with their face supported in the face cradle or built-in face opening of the massage table, thereby allowing the person to receive an oxygen therapy or aromatherapy treatment during a massage.

### BRIEF DESCRIPTION OF THE DRAWINGS

For a fuller understanding of the nature of the present invention, reference should be made to the following detailed description taken in conjunction with the accompanying drawings in which:

FIG. 1 is a perspective view showing the oxygen and aromatherapy delivery apparatus of the present invention installed to the face cradle of a conventional massage table according to one embodiment of the invention, wherein the apparatus is connected to an oxygen supply source such as an oxygen tank or oxygen generator;

FIG. 2 is a side perspective view showing a funnel shaped oxygen and aromatherapy delivery device of the present invention for attachment to a face cradle of a conventional massage table in accordance with the embodiment of FIG. 1;

FIG. 3 is an isolated side elevational view, in partial cross-section, showing the funnel shaped oxygen and aromatherapy device of FIG. 2 fitted to a face cradle of a conventional massage table;

FIG. 4 is a perspective view, shown in cut-away, showing the oxygen and aromatherapy delivery apparatus of FIG. 1 installed on the face cradle of the massage table, and wherein a gap or opening is provided for placement of the user's mouth outside of the top opening of the oxygen and aromatherapy delivery apparatus, thereby allowing the user to breathe in through the nose to inhale the beneficial oxygen and/or aromas, while exhaling out of the mouth exteriorly of the delivery apparatus;

FIG. 5 is a side elevational view showing a user lying face down on a massage table with the oxygen and aromatherapy delivery apparatus installed with the use of a nose mask or other device that is adjustably positionable relative to the face cradle and the user's nose for delivering both oxygen and beneficial aromas to the user while receiving massage therapy; and

FIG. 6 is a side elevational view similar to that shown in FIG. 5, wherein the apparatus of the present invention is connected to just an aromatherapy machine for delivering beneficial aromas to the user, without oxygen, while receiving massage therapy.

Like reference numerals refer to like parts throughout the several views of the drawings.

### DETAILED DESCRIPTION OF THE PREFERRED EMBODIMENTS

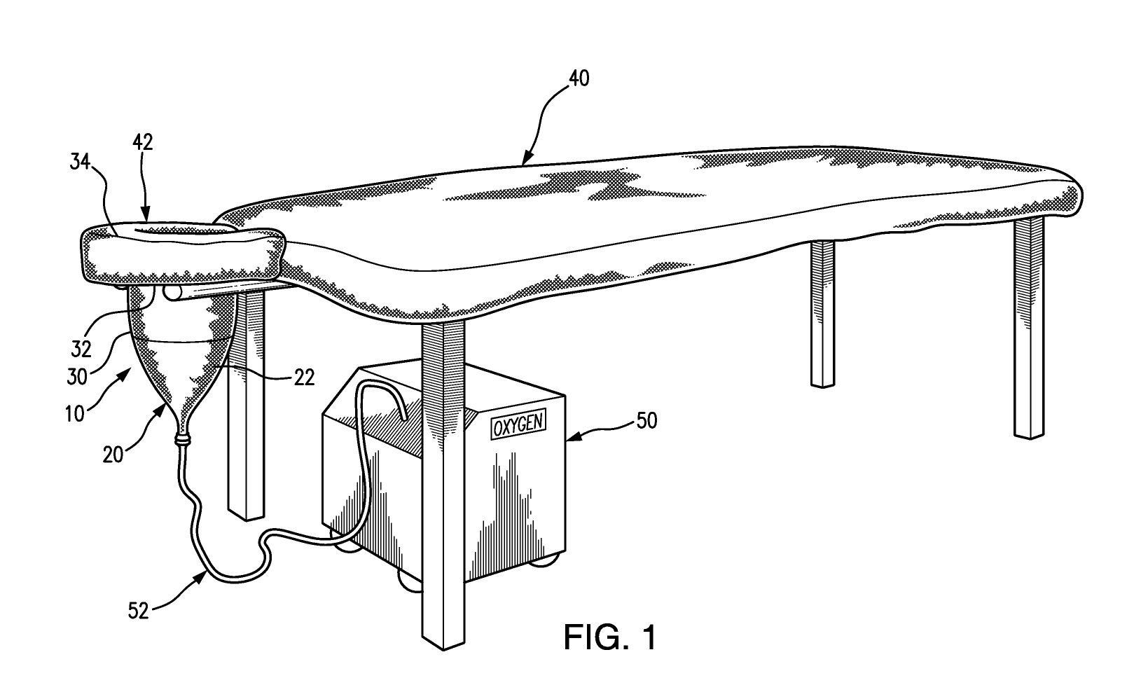

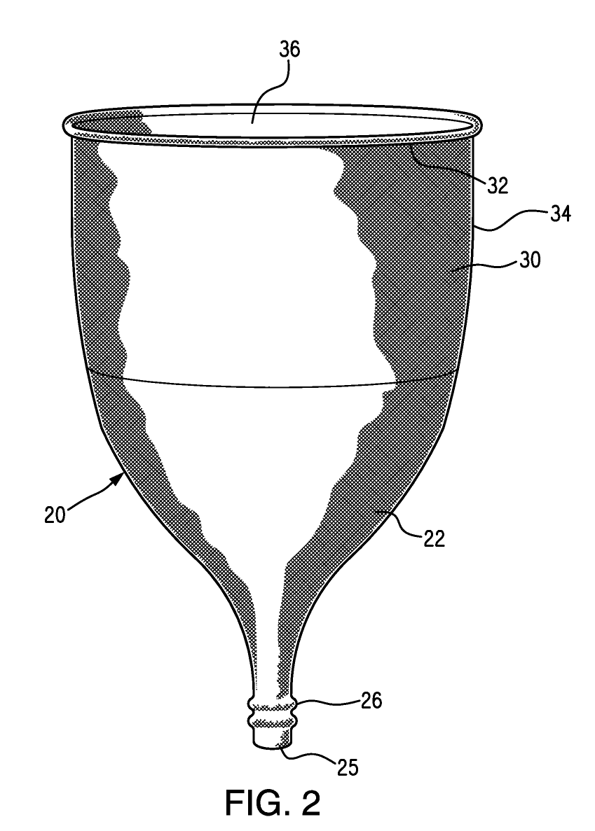

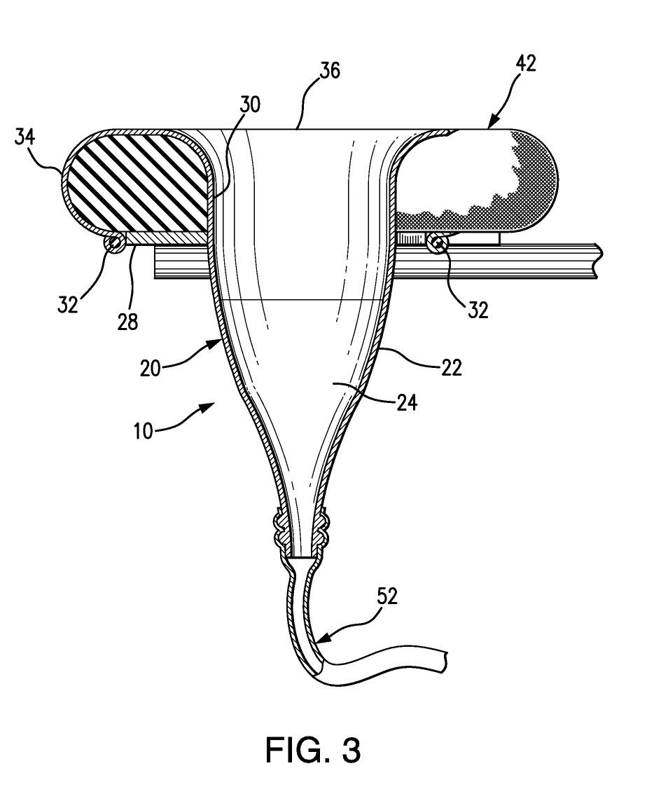

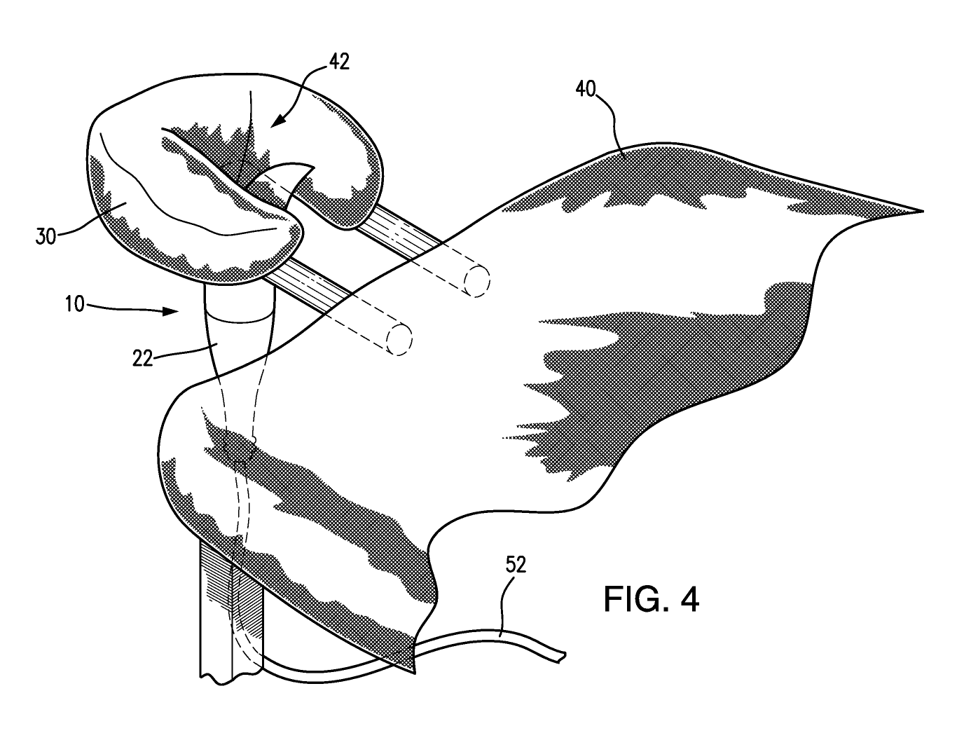

Referring to the several views of the drawings, the oxygen and aromatherapy delivery apparatus is shown and is generally indicated as 10. In one preferred embodiment, the apparatus 10 includes a device 20 that attaches to the face cradle 42 of a conventional massage table 40. The device 20 includes a lower portion 22, generally in the shape of a funnel, that has an interior chamber 24, an opening 25 at a bottom end 26 for connection to a flexible hose 52, and an upper portion 30. As seen in FIGS. 2 and 3, the bottom end 26 may have annular ribs for promoting attachment of the hose 52 thereto. This may be achieved with the use of a rigid (e.g., plastic or metal) insert that is fitted to the material at the bottom end 26. The upper portion 30 is fitted with an annular cover 34 that has a top opening 36 surrounded by an elastic band 32. The cover 34 may be formed of a soft cotton material, similar to a pillowcase. Alternatively, the cover 34 may be formed of another suitable material that can be easily fitted over the sides and above the top of the face cradle 42. To install the device 20 on the face cradle 42, the elastomeric band 32 is stretched in order to enlarge the central opening 36 of the cover 34. This allows the cover 34 to be slipped over the face cradle 42 on the massage table 40 so that the lower funnel shaped portion 22 of the device 20 initially extends above the face cradle 42. The lower portion 22 and bottom end 26 are then tucked through the central opening of the face cradle 42 so that the lower portion 22 and bottom end 26 are positioned below the face cradle 42, as seen in FIGS. 1, 3 and 4. More importantly, the interior chamber 24 of the funnel shaped member 22 is positioned directly below the face cradle 42 and in open communication with the central open area of the face cradle, so that when a person is positioned face down, on the face cradle, with the nose within the open area of the face cradle, the person can easily breathe a steady flow of air from a forced air flow supply source. In a preferred embodiment, the user breathes oxygen and/or the beneficial aromas that are delivered into the interior chamber 24 of the funnel shaped member 22 via the hose 52 that connects to an oxygen supply source 50 (e.g., a pressurized oxygen tank or oxygen generator) and/or an aromatherapy machine 60. The upper portion 30 and particularly the cover 34, is designed to leave an open gap between the face cradle 42 and the table. This open gap is positioned for alignment with the user's mouth when lying face down. In use, the user's nose is positioned directly above the device 10, while the user's mouth is exterior of the device and above the open gap. The user is instructed to breathe in through the nose and exhale from the mouth. This allows the user to breathe the oxygen and/or beneficial aromas through the nose, while exhaling from the mouth exteriorly of the device. This breathing technique also helps relax the user.

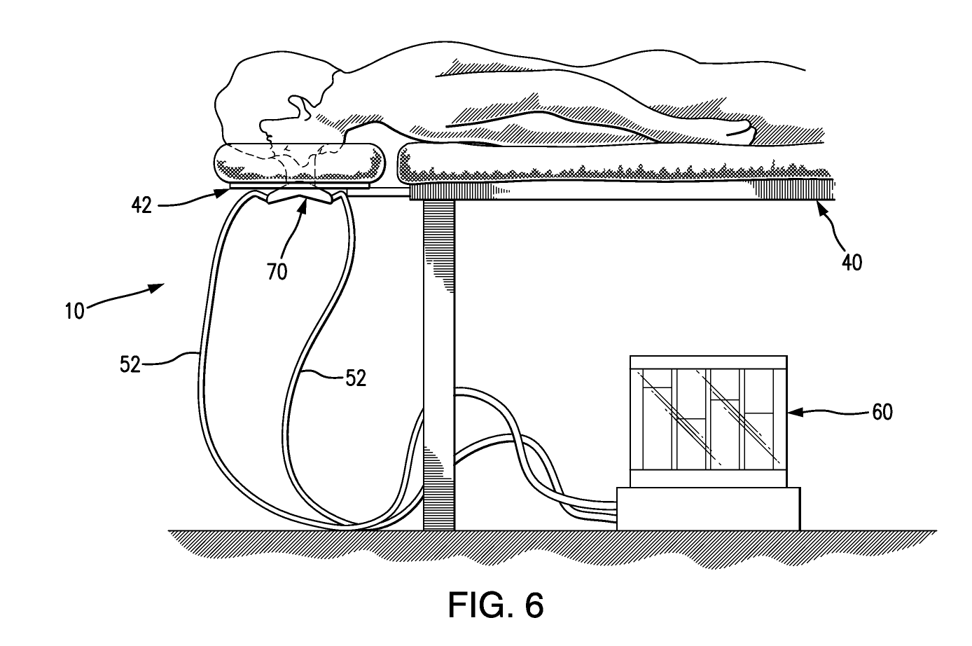

In one embodiment, the flexible hose 52 or other conduit connects between the open bottom end 26 of the funnel shaped member 22 and the oxygen supply source 50, such as an oxygen generating machine. Alternatively, the opposite end of the hose 52 can be connected to an aromatherapy machine 60 (see FIG. 6) that generates aromas from essential oils or oxygen safe scents in order to deliver these aromas for breathing by the person lying on the massage table.

The device 20 that attaches to the face cradle 42 may be made as a disposable product that is replaced after each use. Alternatively, the device 20 may be made so that it can be easily cleaned and disinfected after each use.

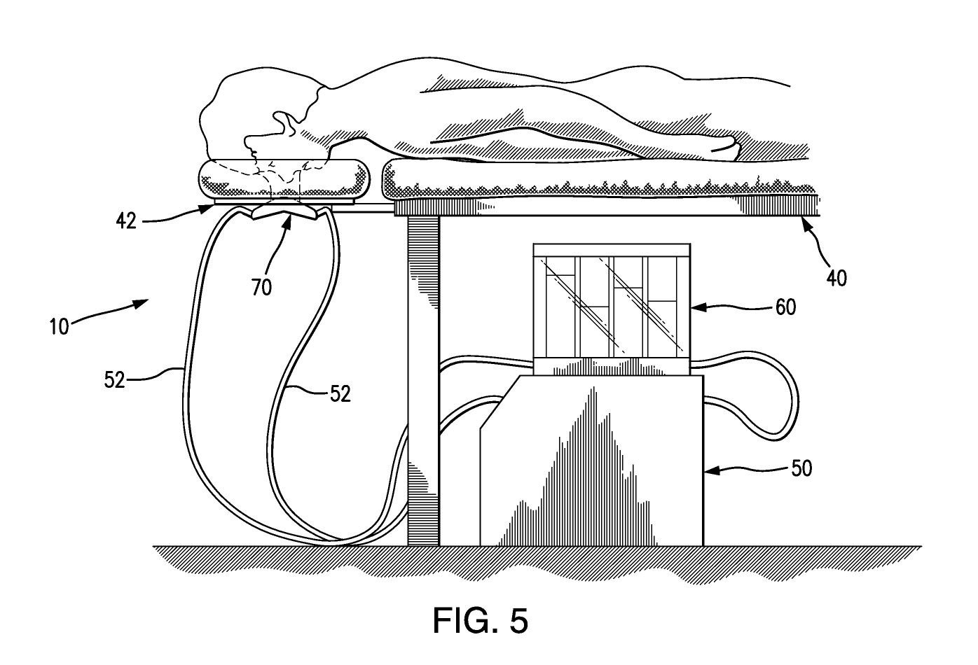

FIGS. 5 and 6 illustrate a further embodiment of the invention for delivering oxygen and/or beneficial aromas to the user while receiving a massage treatment on a massage table. In the embodiment shown in FIGS. 5 and 6, a nose mask 70 or similar device is adjustably fitted to the bottom of the face cradle 42 in direct alignment with the central opening of the face cradle, below the user's nose. Opposite ends of the nose mask 70 connect to hoses 52. The hoses 52 may be connected to the oxygen supply source 50 and/or the aromatherapy machine 60. In FIG. 5, the user is receiving both oxygen and the beneficial aromas from the aromatherapy machine 60. In this instance, oxygen from the oxygen supply source 50 is directed through the aromatherapy machine 60. The user can select one of a plurality of oxygen safe scents that are mixed with the oxygen delivered to the nose mask 70. The user then breathes in the oxygen along with the beneficial aromas. It is important to note that the nose mask 70 or other device does not physically attach to the user's face, head or any other part of the user's body. It is also important to note that the nose mask 70 or other like device may be adjustably fitted to the bottom of either the face cradle 52 or under the massage table 40 in the instance that the massage table 40 has a built-in face opening. As previously noted, some massage tables do not use a face cradle but, instead, have a built-in face opening. The present invention contemplates adjustable attachment of the nose mask 70 or other like device below the built-in opening on such massage tables to achieve the purpose of the invention which is to provide oxygen and/or beneficial aromas of aromatherapy to a user while receiving a massage treatment, and without attaching any kind of mask or device to the user's face, head or body.

FIG. 6 shows the hoses 52 connecting to the opposite ends of the nose mask 70 or other like device and also to the aromatherapy machine 60. In this instance, the user is receiving an aromatherapy treatment, without oxygen, while simultaneously receiving a massage treatment. It is important to note that the arrangement of the oxygen supply source 50 and the aromatherapy machine 60, alone or in combination can be used with either the device 20 shown in FIGS. 1-4 or the nose mask 70 or other like device in any combination. Specifically, the device 20, nose mask 70 or other like device may be connected to just the oxygen supply source 50 as seen in FIG. 1, just the aromatherapy machine 60 as shown in FIG. 6, or both the oxygen supply source 50 and the aromatherapy machine 60 as shown in FIG. 5.

While the present invention has been shown and described in accordance with a preferred and practical embodiment thereof, it is recognized that departures from the instant disclosure are contemplated within the spirit and scope of the present invention.

What is claimed is:

1. An apparatus for use in combination with a massage table that supports a user's face within a face opening communicating with a space below the massage table, said apparatus comprising:
a supply source for releasing a forced flow of air; and
a device positionable below the face opening and in air flow communication with the user's nose when the user's face is positioned in the face opening, and said device being structured and disposed for receiving the forced flow of air from the supply source, and said device being further structured and disposed for directing the forced flow of air towards the user's nose to allow the user to easily breathe the forced supply of air through the user's nose while lying face down on the massage table.

2. The apparatus as recited in claim 1 further comprising:
at least one flexible hose connecting to the device and the supply source for delivering the forced flow of air to the device.

3. The apparatus as recited in claim 2 wherein the supply source is an oxygen supply source that is structured and disposed for releasing a forced flow of air enriched with oxygen.

4. The apparatus as recited in claim 2 wherein the supply source is an aromatherapy machine that is structured and disposed for releasing a forced flow of air enriched with aromas.

5. The apparatus as recited in claim 3 wherein the supply source further includes an aromatherapy machine that is structured and disposed for releasing aromas into the forced flow of air enriched with oxygen.

6. An apparatus comprising:
a support structure for supporting a user in a relaxed position for receiving a massage treatment;
a supply source for releasing a forced flow of air; and
a device positionable in close, spaced relation to the user's nose when the user is supported on the support structure, and said device being structured and disposed for receiving the forced flow of air from the supply source, and said device being further structured and disposed for directing the forced flow of air towards the user's nose to allow the user to easily breathe the forced flow of air through the user's nose while the user is supported on the support structure.

7. The apparatus as recited in claim 6 wherein the supply source is an oxygen supply source that is structured and disposed for releasing a forced flow of air enriched with oxygen.

8. The apparatus as recited in claim 6 wherein the supply source is an aromatherapy machine that is structured and disposed for releasing a forced flow of air enriched with aromas.

9. The apparatus as recited in claim 7 wherein the supply source further includes an aromatherapy machine that is structured and disposed for releasing aromas into the forced flow of air enriched with oxygen.

10. The apparatus as recited in claim 7 further comprising:
at least one flexible hose connecting to the device and the oxygen supply source for delivering the forced flow of air enriched with oxygen to the device.

11. The apparatus as recited in claim 8 further comprising:
at least one flexible hose connecting to the device and the aromatherapy machine for delivering the forced flow of air enriched with aromas to the device.

12. The apparatus as recited in claim 9 further comprising:
at least one flexible hose connecting to the device and the supply source for delivering the forced flow of air enriched with oxygen and the aromas to the device.
